(19) United States
(12) Patent Application Publication
(10) Pub. No.: US 2026/0007406 A1
(43) Pub. Date: Jan. 8, 2026

(54) METHODS AND DEVICES FOR VALVE CLIP EXCISION

(71) Applicant: Evalve, Inc., Santa Clara, CA (US)

(72) Inventor: Alex Khairkhahan, Palo Alto, CA (US)

(21) Appl. No.: 19/301,796

(22) Filed: Aug. 15, 2025

Related U.S. Application Data
(63) Continuation of application No. 16/916,754, filed on Jun. 30, 2020, now Pat. No. 12,408,917.
Application No. 16/916,754 is a division of application No. 15/642,245, filed on Jul. 5, 2017, now Pat. No. 10,736,632.
(60) Provisional application No. 62/359,121, filed on Jul. 6, 2016.
(60) Provisional application No. 62/418,571, filed on Nov. 7, 2016.

(51) Int. Cl. A61B 17/10; A61B 17/00; A61B 17/08; A61B 17/128; A61B 17/22; A61B 17/32; A61B 17/3205; A61B 18/00; A61B 18/14; A61F 2/24

(52) U.S. Cl. CPC A61B 17/10; A61B 17/1285; A61B 17/320016; A61B 17/3205; A61B 18/1492; A61B 2017/00243; A61B 2017/00358; A61B 2017/00783; A61B 2017/00876; A61B 17/083; A61B 2017/22035; A61B 2017/32006; A61B 2018/00369; A61B 2018/00601; A61B 2018/1407; A61B 2018/144; A61F 2/246; A61F 2/2466

(57) ABSTRACT

A system for excising an implanted clip approximating opposed valve leaflets in a heart valve includes a capture catheter configured to be introduced proximate the valve leaflets on one side of the clip, a transfer catheter configured to be introduced proximate the valve leaflets on another side of the clip, and a cutting tool configured to be deployed between the capture and transfer catheters and to be engaged against tissue of at least one of the valve leaflets and to excise the clip. A removal catheter may optionally be used to remove the clip from the heart.

### CROSS-REFERENCE TO RELATED APPLICATION THE INVENTION

The present application is a continuation application of U.S. patent application Ser. No. 16/916,754, filed Jun. 30, 2020, which is a divisional of U.S. patent application Ser. No. 15/642,245, filed Jul. 5, 2017, now U.S. Pat. No. 10,736,632, which claims the benefit of provisional application No. 62/359,121, filed on Jul. 6, 2016, and of provisional application No. 62/418,571, filed on Nov. 7, 2016, the full disclosures of which are incorporated herein by reference.

### BACKGROUND OF THE INVENTION

### 1. The Field of the Invention

The prevent invention relates generally to medical devices and methods. More particularly, the present invention relates to devices, systems, kits, and methods for removing clips and other implanted prostheses from heart valves.

Mitral regurgitation is a commonly encountered valvular disorder and prevalence increases with age. In this condition, blood regurgitates abnormally from the left ventricle into the left atrium during cardiac systole, and this condition can result in numerous adverse consequences such as heart failure due to left ventricular dysfunction, atrial fibrillation, pulmonary hypertension, and death. Published guidelines recommend surgical or transcatheter correction of mitral regurgitation to improve the clinical condition.

Transcatheter correction of mitral regurgitation by implantation of a mitral valve clip (in particular the MitraClip® system from Abbott Vascular) has become a standard therapy for patients at high risk for open surgical corrective procedures. Such clip implantation procedures are performed through a guiding catheter that is inserted into the right femoral vein. One or more mitral valve clips can be delivered through the guiding catheter and implanted to re-approximate the anterior and posterior mitral leaflets (often referred to as an “edge-to-edge” repair). The MitraClip® mitral valve clip is metallic implant made of a cobalt chromium alloy and covered with a fabric mesh.

To date, over 45,000 procedures using the MitraClip® valve clip have been performed globally and there are currently nearly 1000 procedures performed each month. The MitraClip® procedure has been found to be very safe procedure and provides a therapeutic option for patients at high surgical risk.

There are, however, some significant limitations to conventional mitral valve clip technology. First, once a mitral valve clip is implanted, removal typically requires open surgical excision. Second, not all patients achieve satisfactory MR reduction at the time of the procedure due to technical challenges. Finally, up to 1 in 5 patients may have return of significant MR or have need for a repeat intervention within 6 months of the MitraClip® procedure.

If patients have recurrent MR after a mitral valve clip procedure, current options for additional treatment are limited. One option would be to place another mitral valve clip but this is often not possible due to concern for creating mitral stenosis (a mitral valve clip often narrows the valve so that the valve no longer opens properly).

Other options include a variety of mitral valve repair and replacement technologies which are becoming available. Of great interest are transcatheter mitral valve replacement procedures where a bio-prosthetic mitral valve mounted on an expandable frame is deployed in a defective native mitral valve. Such transcatheter “replacement” valves offer complete elimination of mitral regurgitation and would mimic the surgical “gold standard” of mitral valve replacement in selected patients.

At the present time, it can be difficult or impossible to implant a transcatheter mitral valve when a prior mitral valve clip procedure has been performed, since the mitral valve clip(s) interfere and will not allow complete expansion of the new valve. It would therefore be desirable to provide devices, systems, kits, and methods for the transcatheter removal of clips and other implanted prostheses from heart valves. It would be particularly desirable that such devices, systems, kits, and methods for the transcatheter removal of clips and other implanted prostheses from heart valves would leave the heart valve in condition to receive a subsequent transcatheter procedure, such as prosthetic valve implantation, to treat the valve pathology. At least some of these objectives will be met by the inventions described herein.

### 2. Description of the Background Art

See US Patent Publs. 2014/0228871; US2015257883; and US2014135799; U.S. Pat. Nos. 8,500,768; 7,955,340; and 5,895,404; as well as the following guidelines and publications: Nishimura R A, Otto C M, Bonow R O et al. 2014 AHA/ACC guideline for the management of patients with valvular heart disease: executive summary: a report of the American College of Cardiology/American Heart Association Task Force on Practice Guidelines. J Am Coll Cardiol 2014; 63:2438-88; Feldman T, Kar S, Elmariah S et al. Randomized Comparison of Percutaneous Repair and Surgery for Mitral Regurgitation: 5-Year Results of EVEREST II. J Am Coll Cardiol 2015; 66:2844-54; and Maisano F, Alfieri O, Banai S et al. The future of transcatheter mitral valve interventions: Competitive or complementary role of repair vs. replacement?, Eur Heart J 2015; 36:1651-9.

### SUMMARY OF THE INVENTION

The present invention provides transcatheter tools and procedures that can be used to separate and optionally remove one or more mitral valve clips from native mitral leaflet tissue. In such procedures, the physician can insert specifically designed catheters either from the left atrium or left ventricle. A guidewire may be placed around the leaflet and valvular tissue adjacent to the previously implanted mitral valve clip. Utilizing mechanical cutting or abrasion, or radiofrequency energy, the mitral valve clips may be excised from either the anterior or posterior leaflets, or both, restoring the native valve orifice and allowing future repair or replacement therapy. After separation of the mitral valve clips from native leaflet tissue, the degree of mitral regurgitation would be expected to worsen acutely. The separation procedure of the present invention in contrast can be performed immediately prior to transcatheter valve implantation or another transcatheter corrective procedure. For instance, if transapical access were used for deployment of a new transcatheter mitral valve, then transseptal access could be used to release the mitral valve clips, or vice versa.

In a first aspect of the present invention, a method for excising a clip approximating an opposed pair of valve leaflets in a heart valve comprises introducing a capture catheter into a heart chamber adjacent to the valve leaflets on one side of the clip. A transfer catheter is also introduced into the heart chamber adjacent to the valve leaflets on another side of the clip, and a cutting member is deployed between the transfer catheter and the capture catheter to place or engage the cutting member against tissue of at least one of the valve leaflets. The cutting number is then used to excise the clip from at least one of the valve leaflets to release fixation and allow separation of the valve leaflets.

The excision methods of the present invention are useful on any heart valve where a clip may have been placed to enhance coaptation or for other reasons. Most typically, the heart valve is a mitral valve, and the catheters may be introduced transseptally into the left atrium above the valve or may introduced transapically into the left ventricle beneath the mitral valve. In other instances, the target heart valve can be a tricuspid valve, and the catheters may be introduced into the right atrium above the tricuspid valve or into the right ventricle below the tricuspid valve.

In exemplary embodiments, deploying the cutting member comprises steering a tip of at least one and usually both of the capture catheter and the transfer catheter to bring the tips of both catheters into proximity with the valve clip. Typically, at least one of the capture catheter and transfer catheter may be advanced through an opening between the valve leaflets adjacent to the fixed valve clip, where the advanced catheter may then be steered around the opposite side of the valve clip and brought up through the space between the valve leaflets on an opposite side of the valve clip. The cutting member may then be passed between the transfer catheter and the capture catheter to span a target region of the valve leaflet to be excised. The catheters may then be manipulated to extend the cutting member past a side of the valve clip so that the cutting member is positioned to excise a portion of the valve leaflet on one side of the valve. As will be described in more detail below, the valve clip may be left implanted in the opposed valve leaflet, and a subsequent valve intervention procedure performed with the clip left attached to the opposed valve leaflet. Alternatively, the capture and transfer catheters may be used to reposition the cutting member on the other side of the valve clip so that the opposed leaflet may be excised from the clip to completely release the clip and allow the clip to be removed from the heart prior to performing a subsequent intervention on the heart valve. When releasing and removing the valve clip, it will of course be necessary that the valve clip be constrained, typically by a removal catheter as described herein below.

In still further specific examples, the cutting member may be deployed by engaging a magnetic element on the capture catheter against a magnetic element on the cutting member. The magnetic elements are typically at the tip of the capture catheter and an end of the cutting member, and the catheter(s) may be manipulated to draw the cutting member past either or both sides of the valve clip in order to excise of the valve leaflet tissue to release the clip.

In an alternative embodiment, the cutting element may comprise a loop which is used to capture a free end of the cutting member, and the capture catheter may then be manipulated to advance the loop over a free end of the cutting member and draw the cutting member past the clip through the valve tissue.

In still further embodiments, deploying the cutting member may comprise drawing the tips of the capture catheter and the transfer catheter together to form a path past the valve clip and advancing the element over the path. After such advancement and positioning of the cutting member, the capture catheter and/or the transfer catheter may be manipulated to manipulate the cutting member to excise the valve tissue to release the clip.

As discussed above, in some embodiments of the methods of the present invention, the clip(s) will be excised from one valve leaflet only, leaving the clip(s) implanted in an opposed valve leaflet. Freeing of the clip(s) from at least one valve leaflet will in at least some cases be sufficient to release the opposed valve leaflets and enable performance of the subsequent valve replacement or other interventional procedure. In other instances, however, it will be preferable to completely remove the valve clip(s) from the heart valve prior to performing a subsequent valve implantation or other intervention. In such cases, a removal catheter or other device will typically be used to constrain or capture the clip(s) during the excision and withdraw the excised clip(s) from the heart chamber prior to any further intervention.

In still other specific embodiments of the methods of the present invention, the clip(s) may be excised from the valve leaflet tissue in a variety of ways. For example, the cutting member may comprise an electrode segment or other electrically conductive region capable of delivering a radiofrequency (RF) cutting current to the tissue. In particular, the RF current would be delivered in a cutting mode so that the cutting member could excise the valve tissue adjacent to the valve clip. In other instances, the cutting member could comprise a sharpened or abrasive region which could be used to mechanically cut through the valve tissue. For example, a sharpened or abrasive cutting region could be reciprocated so that it would “saw” through the tissue in order to excise the valve. Other known tissue cutting modalities could also be employed.

As described thus far, the capture catheter, transfer catheter, and cutting member are used to excise the tissue from a single valve leaflet, leaving the valve clip attached to the opposed valve leaflet. While removing the valve from a single leaflet will allow sufficient opening of the leaflets for subsequent prosthetic valve implantation or other corrective procedures, it will sometimes be desirable to remove the valve clip from both opposed valve leaflets. In such instances, it will be preferred to introduce a clip removal catheter into the heart chamber in order to stabilize the eventually remove the valve clip. A distal end of the clip removal catheter can be engaged against the clip and will usually be attached to the clip while the clip is excised from one or both valve leaflets using the cutting member.

In still further exemplary embodiments, the capture catheter, the transfer catheter, and optionally the clip removal catheter can be introduced transseptally, usually simultaneously through a transseptal catheter or sheath. In still other specific embodiments, the capture catheter, the transfer catheter, and optionally the clip removal catheter can be introduced transapically, typically simultaneously through a transapical sheath or catheter.

In a second aspect of the present invention, a system for excising an implanted clip approximating opposed valve leaflets in the heart valve comprises a capture catheter, a transfer catheter, and a cutting member. The capture catheter is configured to be introduced into a heart chamber adjacent to the valve leaflets on one side of the clip. The transfer catheter is configured to be introduced into the heart chamber adjacent to the valve leaflets on another side of the clip, and the cutting member is configured to be deployed from the transfer catheter to the capture catheter to place the cutting element against tissue of at least one of the valve leaflets to excise the clip.

In specific aspects of the system, at least one of the capture catheter and the transfer catheter will have a steerable tip, usually both having a steerable tip. The capture catheter will often have a magnetic distal tip configured to engage and capture a magnetic element disposed at the distal end of the cutting member. In this way, the magnetic tip on the capture catheter can be used to attract and engage the magnetic end of the cutting member so that the cutting member can be deployed between the capture catheter and the transfer catheter. Thus, a cutting region of the cutting member can be positioned to engage the valve leaflet tissue adjacent to the valve clip so that the cutting region can be used to cut the tissue and excise the clip. For example, the cutting region could comprise a sharpened or abrasive region which can be used to mechanically cut the valve tissue. Alternatively, the cutting region could comprise an RF electrode which can be powered with a cutting current to excise the valve leaflet tissue adjacent to the clip.

The systems of the present invention may further comprise an introductory sheath, typically a transseptal sheath or a transapical sheath, for delivering the catheters of the present invention to a heart chamber. Usually, the introductory sheath will be sufficiently large to accommodate at least the capture catheter and the transfer catheter simultaneously. Often times, the system will further comprise a removal catheter, and the introductory sheath will be sized to be sufficiently large to simultaneously accommodate each of the capture catheter, transfer catheter, and removal catheter.

The clip removal catheter of the present invention will typically have a distal end configured to engage and capture the clip Certain valve clips, such as the MitraClip® valve clip, have a unique shape that provides a “docking” feature which can be used by the clip removal and optionally other catheters of the present invention to “dock” with either the atrial or ventricular aspect of the valve clip. The shape of the valve clip(s) is typically easy to discern on fluoroscopy which facilitates targeting and optionally docking a removal or other catheter with the clip during a removal procedure. Such docking has several advantages. First, docking can stabilize the valve clip relative to the catheter system which is advantageous as the valves are moving during the beating heart procedure. Second, docking facilitates orienting the catheter(s) to improve position of the cutting member, e.g. looping a cutting wire so that it encircles the clip using either the magnetic or snare techniques shown above. Third, by docking a removal or other catheter with the valve clip, the catheter and cutting member can be stably positioned in close proximity to the valve clip, thus minimizing the risk of entangling or snaring the chordal apparatus with the loop wire or other cutting member. Fourth, docking can be performed either from an atrial or a ventricular aspect. For example, atrial docking can be achieved by docking into a groove on the top of a “Y” portion of the MitraClip® clip. Ventricular docking may be achieved by docking with the bottom of the “Y” portion of the MitraClip® clip. The docking can be passive, e.g. via an interference fit, or can be active where the removal or other catheter has a “grasping” feature to securely attach the MitraClip® clip. Docking can be performed on each valve clip individually or on several at the same time with modification in the docking feature.

The removal catheters of the present invention may be designed to dock with the MitraClip® or other valve clips to place a snare around the clip. After the clip is freed from both the anterior and posterior leaflets (or septal leaflet in case of a tricuspid valve) using the previously described catheters, the clip can be removed from the body through the access sheath by the removal catheter. The removal catheter may have any one of a variety of clip-grasping features, such as a snare, biopsy-type forceps, or jaws that secure the valve clip to hold it after it has been freed from both leaflets so it can be removed from the body. The capture and/or transfer catheter may also be configured to dock with the valve clip, e.g. have a distal tip with a shape complementary to the shape of the valve clip. The catheter that removes the clip can be placed via a transvenous transseptal approach or left ventricular transapical approach. In some instances, a removal catheter can be designed with a funnel shaped tip to allow the valve clip to be drawn into the catheter with proper orientation.

The catheter systems of the present invention can also be used to encircle and remove pacemaker or defibrillator leads. For example, by sliding a loop encircling the lead along a length of the lead, the lead can be excised from the heart or vascular tissue in which it has been implanted. In particular, the catheters of the present invention can be used to free leads from vascular venous tissue such as the axillary vein, brachiocephalic vein, and/or superior vena cava. The catheters of the present invention can also be used to free leads from a tricuspid valve or the right ventricular myocardium. In particular, a lead may be freed from the tricuspid valve in situations where the lead may be causing tricuspid regurgitation (TR). The lead could optionally be repositioned to reduce TR. The catheters can also be used to free a lead to allow the lead to be repositioned in the tricuspid valve orifice as desired to facilitate placement of a transcatheter valve in the tricuspid position.

The novel features believed characteristic of the illustrative examples are set forth in the appended claims. The illustrative examples, however, will best be understood by reference to the following detailed description of an illustrative example of the present disclosure when read in conjunction with the accompanying drawings, wherein:

### BRIEF DESCRIPTION OF THE DRAWINGS

FIGS. 1 and 1A illustrate the anatomy of the mitral valve after implantation of a mitral valve clip.

FIGS. 2A and 2B illustrate a capture catheter constructed in accordance with the principles of the present invention with a steerable distal region shown in its straightened configuration (FIG. 2A) and in deflected configurations (FIG. 2B).

FIG. 3 illustrates a transfer catheter constructed in accordance with the principles of the present invention.

FIG. 4 illustrates a cutting member used with the capture and transfer catheters in the methods of the present invention.

FIGS. 5A through 5E illustrate different embodiments of magnetic tips on the capture catheters and magnetic distal elements on the cutting members.

FIG. 6 illustrates an alternative system according to the present invention including a capture catheter having a capture loop at its distal end.

FIGS. 7A through 7F illustrate use of a capture catheter and a transfer catheter in a method for removing a pair of clips implanted in a mitral valve in accordance with the principles of the present invention.

FIG. 8 illustrates transapical introduction of a capture catheter and a transfer catheter into a left atrium for excising a valve clip in a mitral valve in accordance with the principles of the present invention.

FIGS. 9A through 9C illustrate use of a capture catheter and a transfer catheter in a method for removing a valve clip from a tricuspid valve in accordance with the principles of the present invention.

FIGS. 10A through 10E illustrate use of a capture catheter and a transfer catheter in a method for removing a single valve clip from a mitral valve in accordance with the principles of the present invention.

### DETAILED DESCRIPTION OF THE INVENTION

The present invention provides apparatus and methods for the separation of one or more valve clips from mitral and other heart valve leaflets. The valve clips can be located anywhere along the valve coaptation plane (central, medial, lateral, or commissural), and in some embodiments the valve clips are removed from one of the coapting valve leaflets while being left in placed in the other of the coapting valve leaflets. In other embodiments, the valve clips will be removed from both valve leaflets and be extracted entirely from the heart.

The technology of the present invention can be deployed from a left atrial aspect (transseptal or direct atrial access), or from the left ventricular aspect (transapical, direct ventricular puncture, or retrograde aortic access). The cutting action can be provided by a “noose,” by a scissor-like device, by a wire-based cutter similar to a “cheese cutter,” by a radiofrequency electrode cutting element, or the like. In some instances, the mitral valve clips may be removed from both anterior and posterior leaflets, and a snare, bioptome-type device, or other removal catheter may be used to completely remove the mitral valve clip from the body. The present invention may be used to remove valve clips from tricuspid valves as well as mitral valves, and the catheters be placed through transseptal and transapical guiding catheter which are typically large enough to accommodate all catheters being used in the procedure simultaneously.

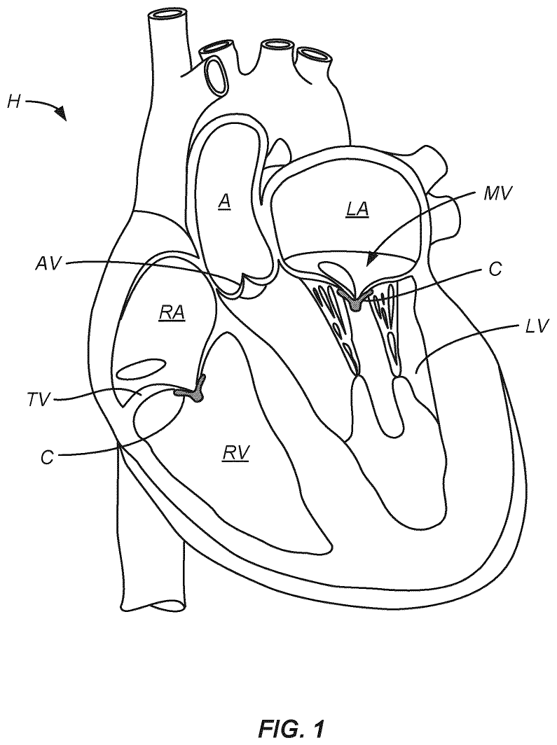

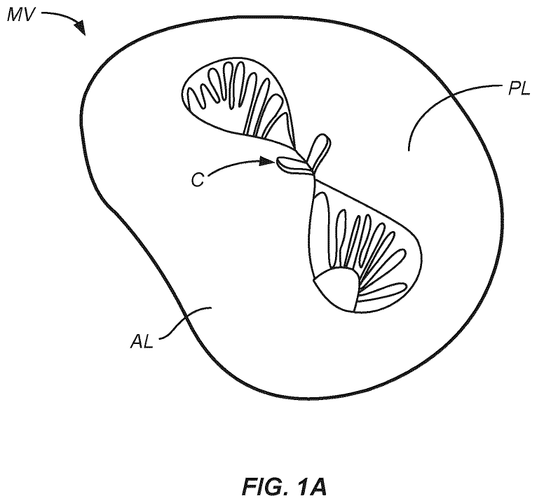

Referring now to FIGS. 1 and 1A, valve clips C may be implanted in the leaflets of a mitral valve MV as well as a tricuspid valve TV in a heart H. The mitral valve MV separates the left atrium LA from the left ventricle LV, and the tricuspid valve TV separates the right atrium RA from the right ventricle RV. For completeness, it is noted that blood flows from the left ventricle LV through the aortic valve AV into the aorta A. As shown in more detail in FIG. 1A, the clip C in the mitral valve MV may be located near a middle of the valve opening between the anterior leaflet AL and the posterior leaflet PL. The clip C, however, could also be implanted at other locations between the leaflets away from the middle of the valve opening.

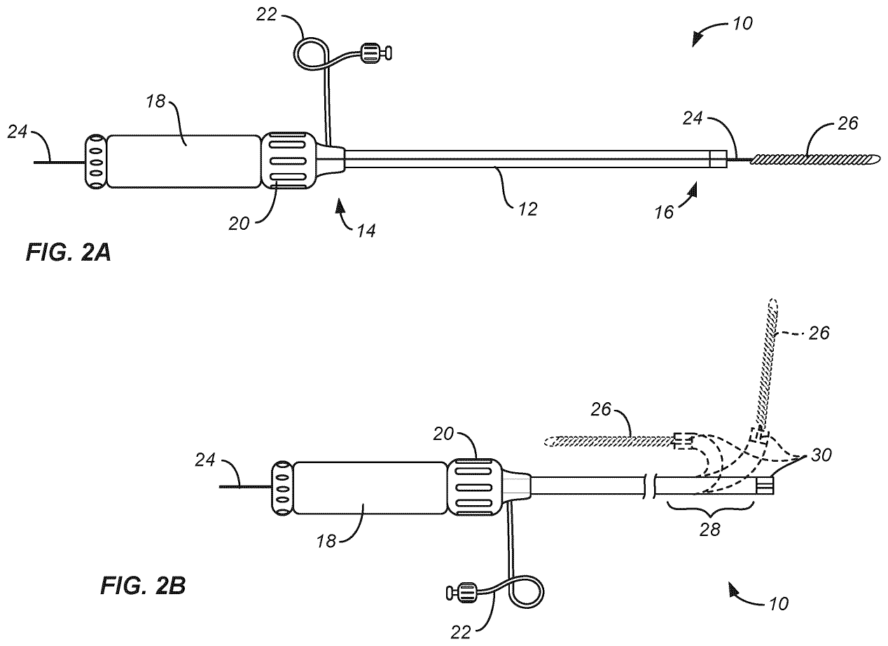

Referring now to FIGS. 2A and 2B, a capture catheter 10 comprises a shaft 12 having a proximal end 14 and a distal end 16. A control handle 18 is attached to the proximal end of the shaft, and a deflection knob 20 is located at the distal of the handle. The capture catheter 10 will typically also include a flush port 22 and will have a central lumen adapted to receive a guidewire 24 having a shapeable tip 26 at its distal end. As shown in particular FIG. 2B, a distal region 28 of the catheter shaft 12 will be actively deflectable or “steerable” so that the region can be deflected by up to 180°, as shown. Such deflection will be controlled by the deflection knob 20, and suitable deflection mechanisms may include pull wires, slotted regions at the distal end of the shaft, or any other conventional catheter deflection technology.

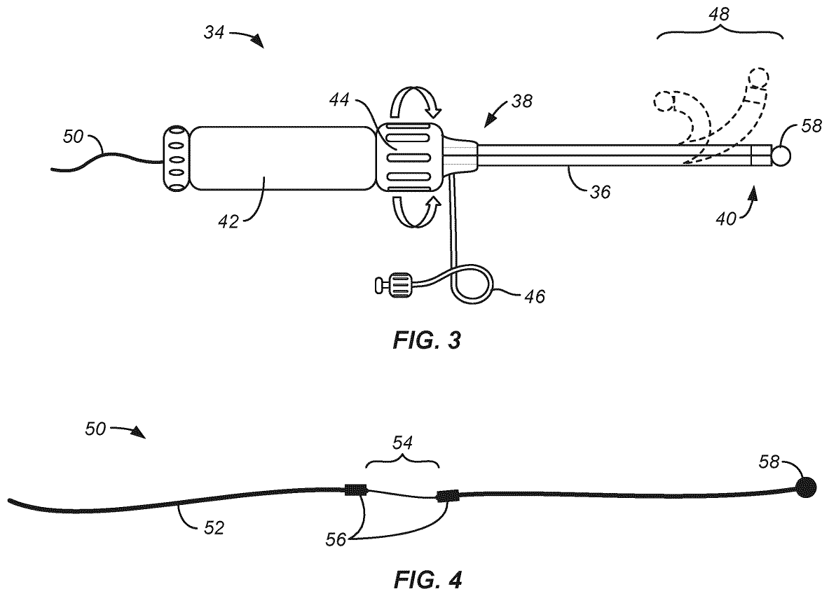

Referring now to FIG. 3, a transfer catheter 34 includes a shaft 36 having a proximal end 38 and a distal end 40. A control handle 42 is attached to the proximal end of the shaft, and a deflection knob 44 is disposed at the distal end of the control handle 42. The transfer catheter 34 may also include a flush port 46 and will typically have a steerable distal tip 48 similar to that described above for the capture catheter 10.

Referring now to FIG. 4, a cutting member 50 comprises an elongate body 52 which typically has a wire-like configuration (typically being electrically conductive in the case of electrosurgical cutting members as described below) so that it may be fed through an advancement lumen of the transfer catheter 34. The cutting member 50 further includes a cutting region 54 which typically spans a region near a middle of the length of the elongate body, typically having a length from 0.5 cm to several centimeters, and which typically is bounded on each side by a radiopaque marker 56. In the case of electrosurgical cutting members, the member may comprise an electrically conductive wire where the cutting region uninsulated and the regions on either side of the cutting region are electrically insulated. A magnetic distal element 58 will typically be located at one end of the elongate body 52 and, as shown in FIG. 3, when the cutting member 50 is loaded onto the transfer catheter 34, the magnetic distal element 58 will extend outwardly from the distal tip 40 of the catheter so that it is exposed and available to magnetically attract the magnetic tip 30 of the capture catheter 10 as described below.

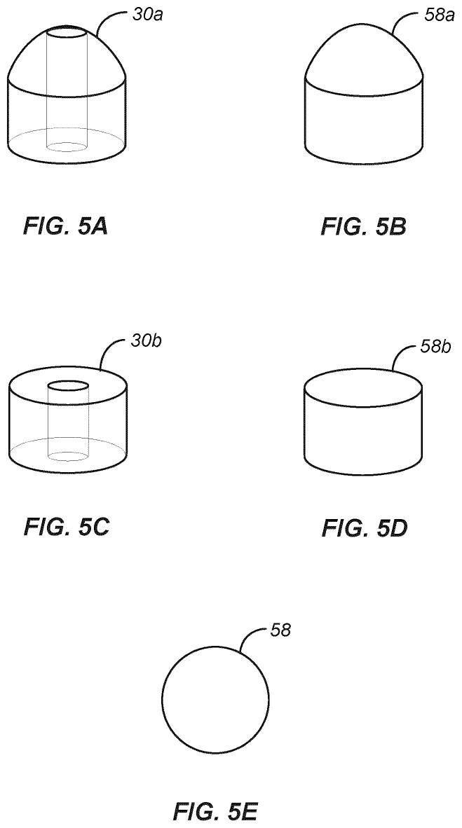

As shown in FIGS. 5A through 5E, the magnetic tip 30 of the capture catheter 10 may take a variety of forms. Magnetic tip 30a (FIG. 5A) has a bullet shape with a through lumen to accommodate the guidewire of the capture catheter. Magnetic tip 30b has a disc-like shape and also has a through lumen to accommodate the guidewire in the capture catheter.

The magnetic distal element 58 of the cutting member 50 may also have a variety of configurations. Most simply, the magnetic distal element 58 will have a sphere shape as shown in FIG. 5E. Alternatively, the magnetic distal element may have a bullet shape 58a as shown in FIG. 5B or a disc-like shape 58b as shown in FIG. 5D.

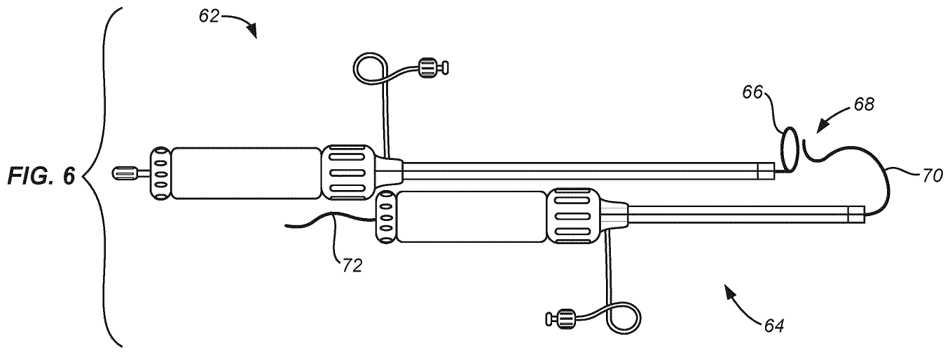

As described thus far, the capture catheter 10 and the cutting member 50 will each have magnetic elements to allow capture of the cutting member by the capture catheter 50. While this is a preferred design, a variety of other capture mechanisms could also be employed. For example, as shown in FIG. 6, a capture catheter 62 may carry a capture loop 66 at its distal end. The capture loop could be used then to capture a free end 70 of a cutting member 72 carried by transfer catheter 64. The use of hooks, barbs, coil targets, and the like, would also be possible to allow for capture of the cutting member by a capture catheter.

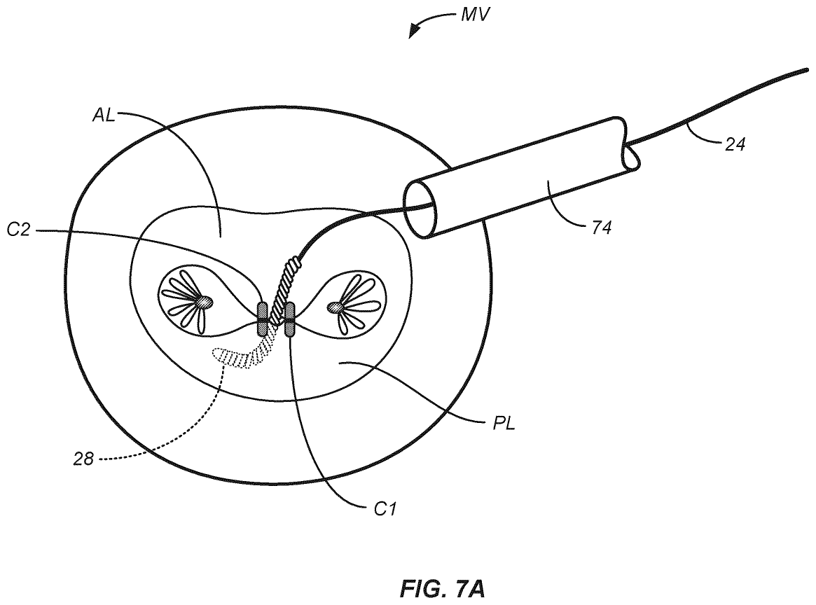

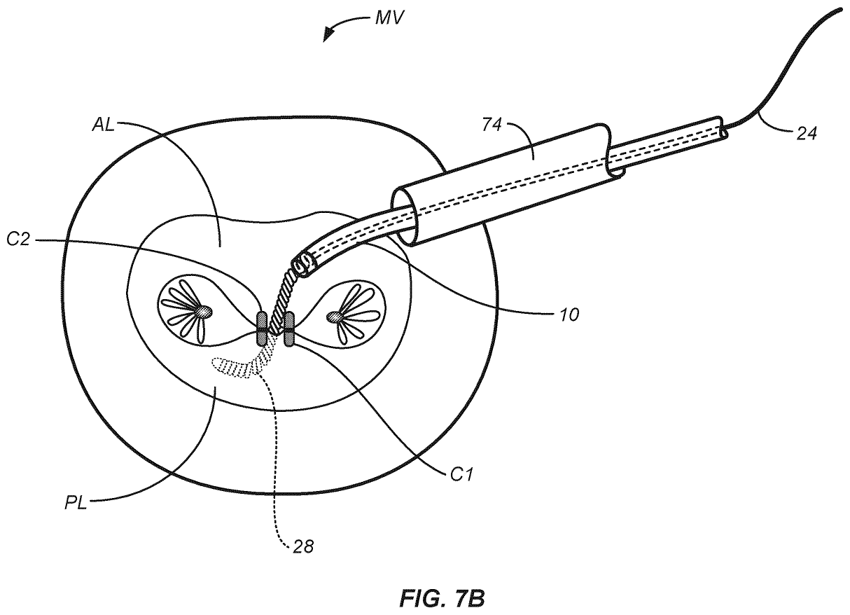

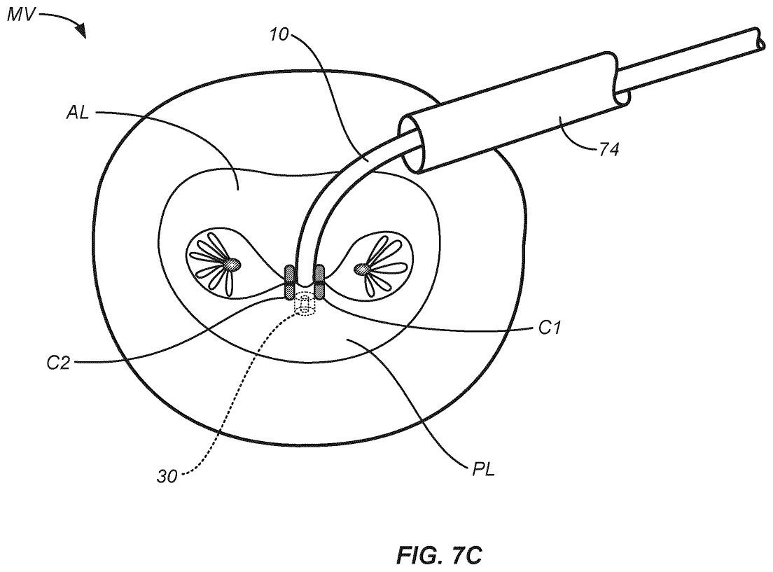

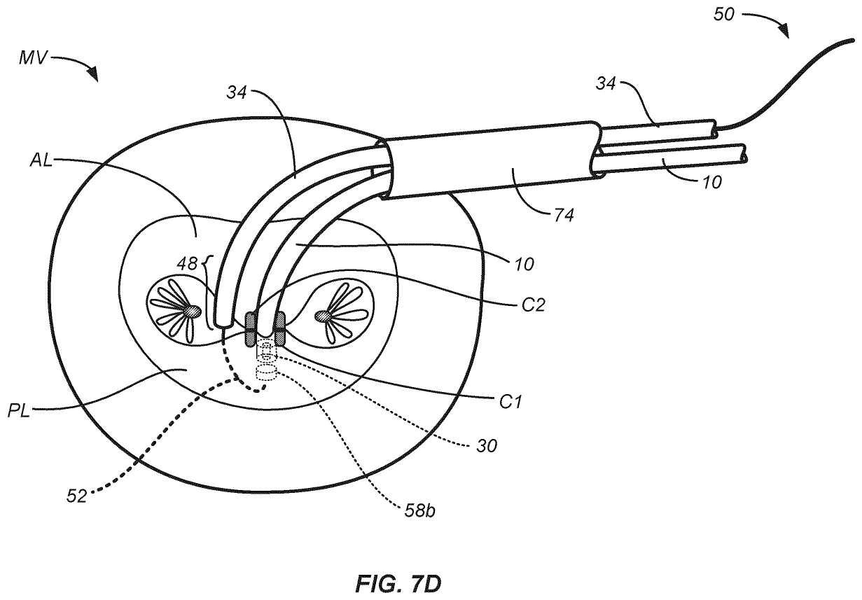

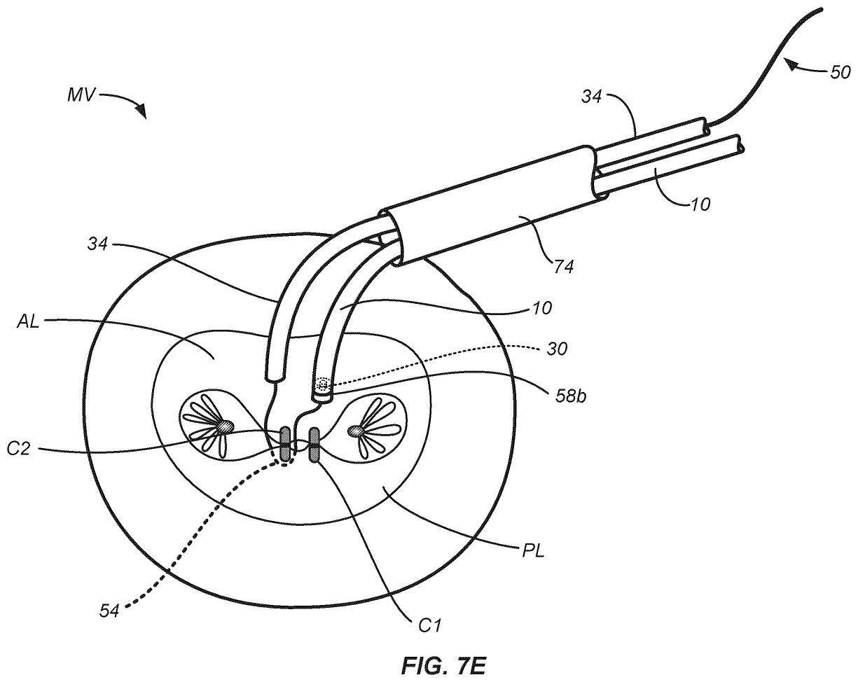

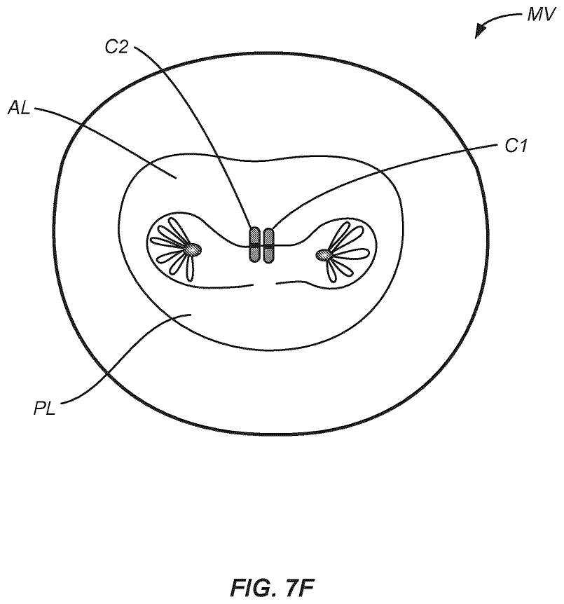

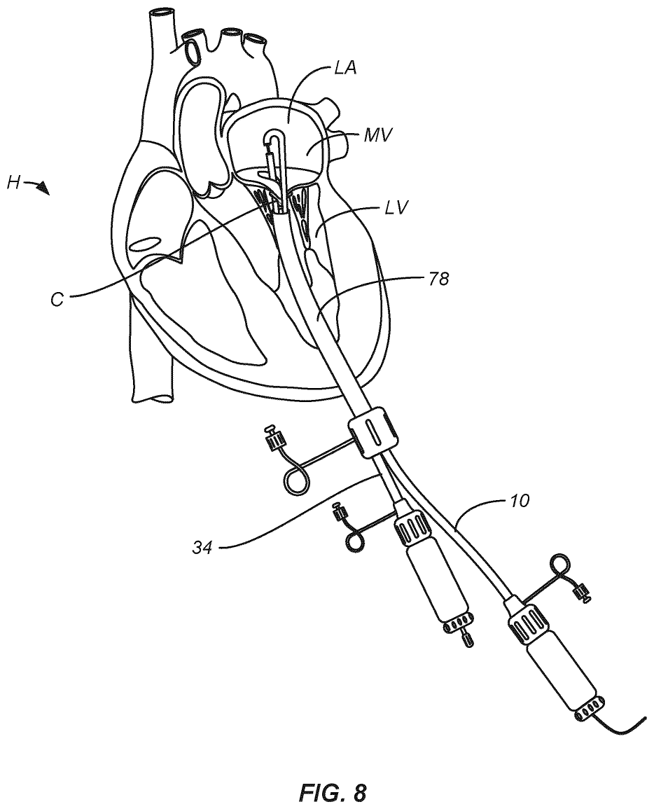

Referring now to FIGS. 7A through 7F, the use of capture catheter 10 and transfer catheter 34 for removing a pair of clips C1 and C2 from a mitral valve MV via a transseptal approach will be described. A steerable distal tip 28 of guidewire 24 is first introduced through a transseptal sheath 74 into a left atrium above the mitral valve. The tip 28 can be advanced between the clips C1 and C2 under fluoroscopic guidance with the heart beating. Optionally, the capture catheter 10 could be used to assist in placing the guide wire 24 between the clips before the catheter 10 is the advanced over the guide wire. The guide wire can be passive or active.

After the guidewire is placed, as shown in FIG. 7A, the capture catheter 10 can be advanced over the guidewire 24 so that it is positioned above the clips C1 and C2, as shown in FIG. 7B. The distal end of the capture catheter 10 is then advanced between the clips C1 and C2 so that the magnetic tip enters the left ventricle, as shown in FIG. 7C. The transfer catheter 34 may then be introduced through the transseptal sheath 74 in parallel to the capture catheter 10 and steered to advance the magnetic distal element 58b on the cutting member 50 so that it engages the magnetic tip 30 on the capture catheter 10. For example, the steerable distal region 48 of the transfer catheter 34 can be advanced downwardly between the anterior leaflet AL and posterior leaflet PL and manipulated to engage the magnetic distal element 58b against the magnetic tip 30.

After the cutting member 50 and capture catheter 10 are coupled together, the capture catheter 10 may be pulled back to expose the cutting region 54, and the catheters manipulated to engage the cutting region 54 against the second valve clip C2, as shown in FIG. 7E. The cutting region 54 can then be manipulated to saw through or electrode surgically cut the tissue of the posterior leaflet PL surrounding the clip C2. The procedure may then be repeated to excise the first clip C1 from the posterior leaflet PL, as shown in FIG. 7F, resulting in opening of the mitral valve to allow a prosthetic valve implantation or other intervention to take place. While excision of the posterior leaflet is illustrated in FIGS. 7A-7F, in some instances it would be preferable to excise the anterior leaflet tissue leaving the clip(s) attached to the posterior leaflet.

Referring now to FIG. 8, excision of valve clip C from the mitral valve MV can also be performed using capture catheter 10 and transfer catheter 34 through a transapical sheath 78. Using the transapical approach, the distal tips of the catheters 10 and 34 will be advanced upwardly from the left ventricle LV to the left atrium LA, and the tissue can then be excised using the catheters as generally as described previously.

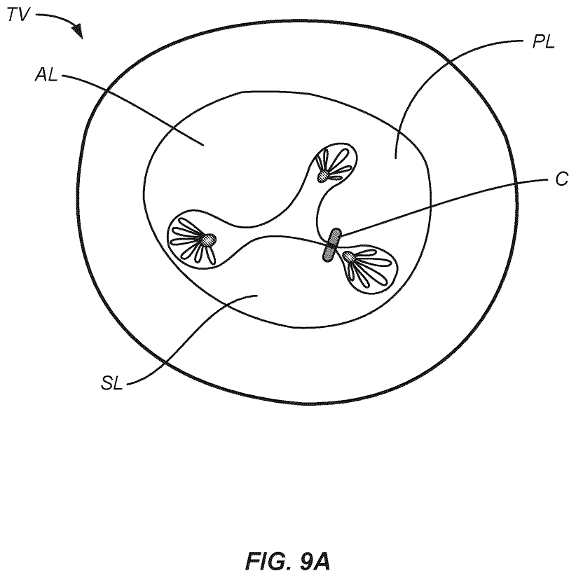

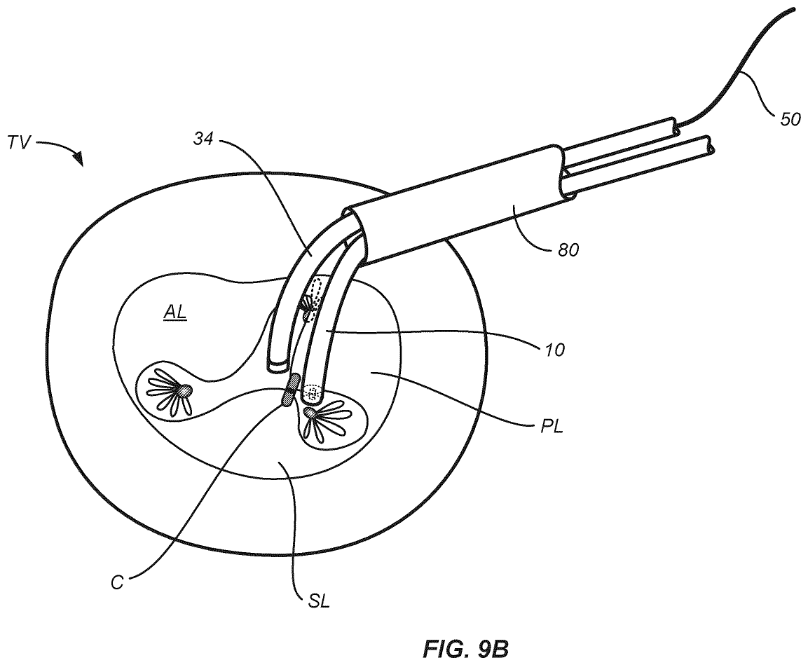

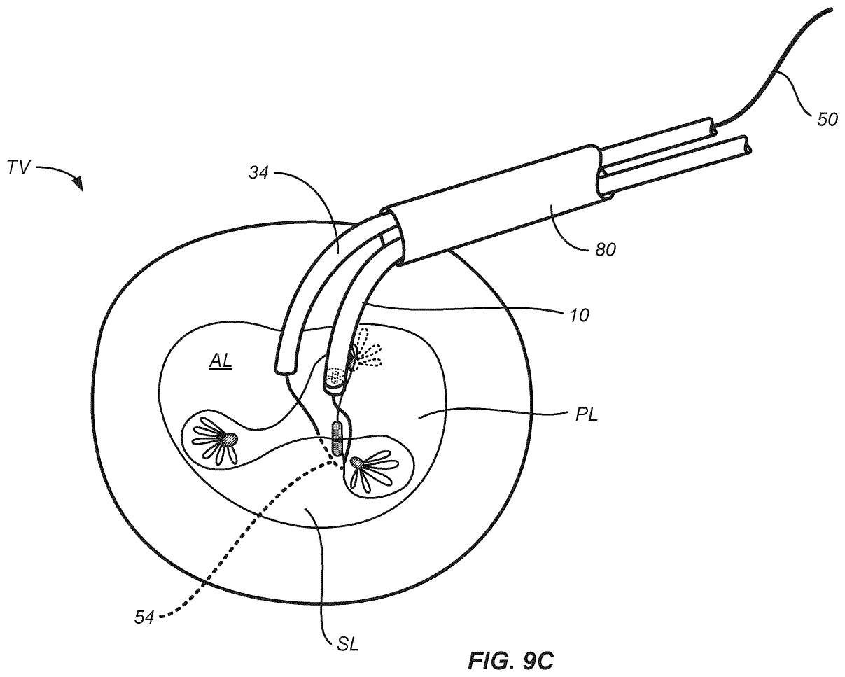

Referring now to FIGS. 9A through 9C, excision of a valve clip C implanted between an anterior leaflet AL and a septal leaflet SL in a tricuspid valve TV will be described. Capture catheter 10 and transfer catheter 34 can be introduced through an access sheath 80 into the right atrium above the tricuspid valve, as shown in FIG. 9B. The catheters 10 and 34 can be used to position the cutting region 54 of the cutting member 50 on a side of the clip C in the septal leaflet SL as shown in FIG. 9C. The cutting member 50 can then be manipulated in any of the ways described previously to excise the tissue of the septal leaflet SL to release the clip C and open the tricuspid valve, as shown in FIG. 9C. The portion of the clip C implanted in the anterior leaflet can also be excised, or the clip can be left in place, prior to performing a subsequent intervention on the valve.

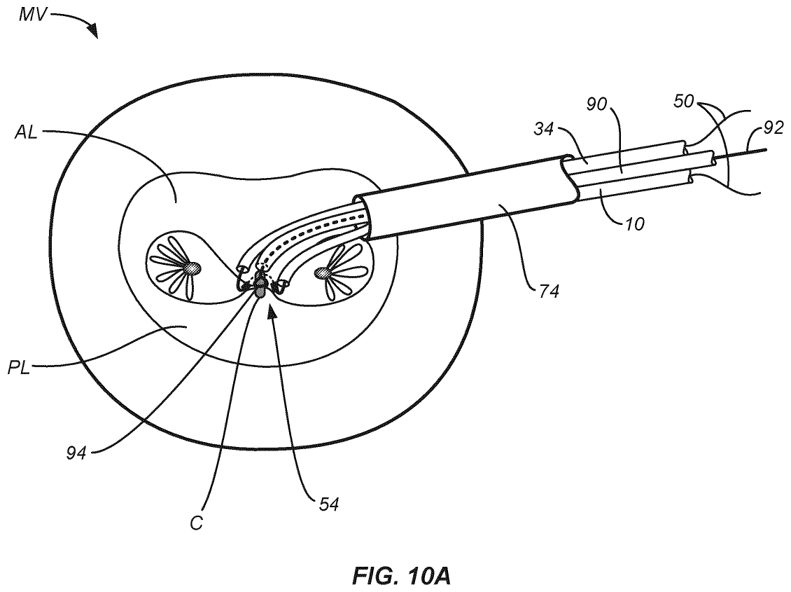

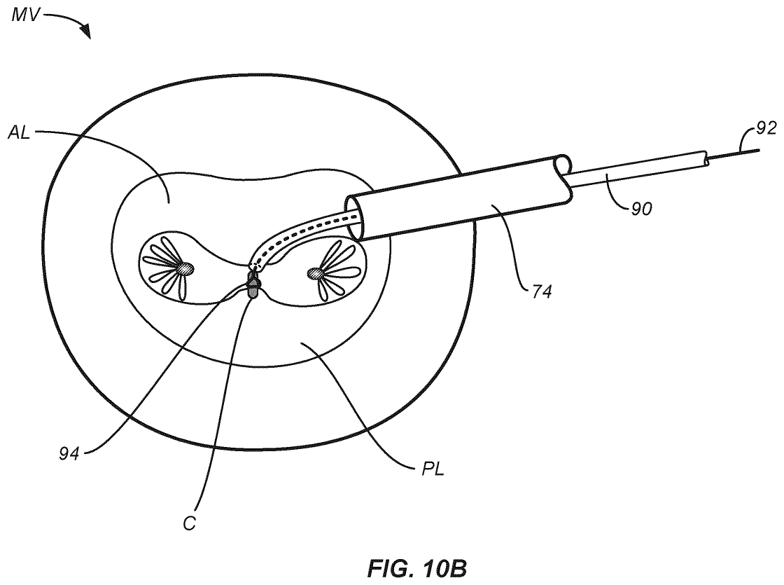

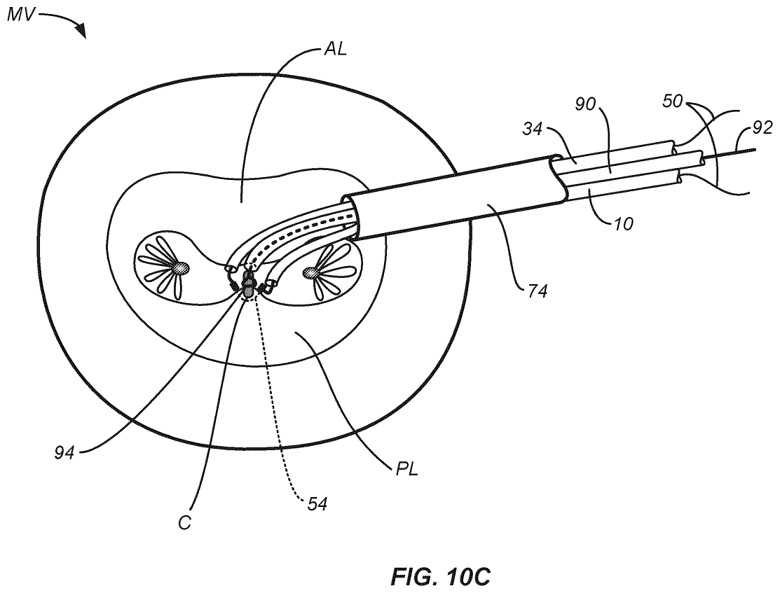

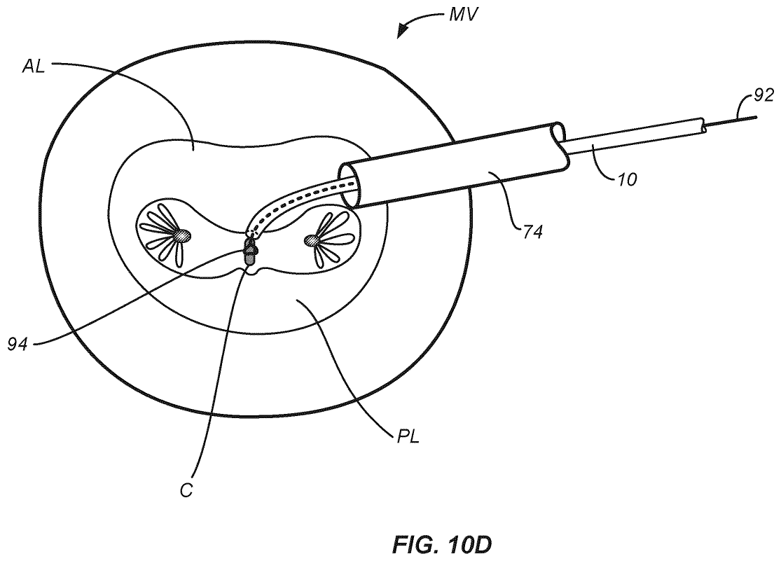

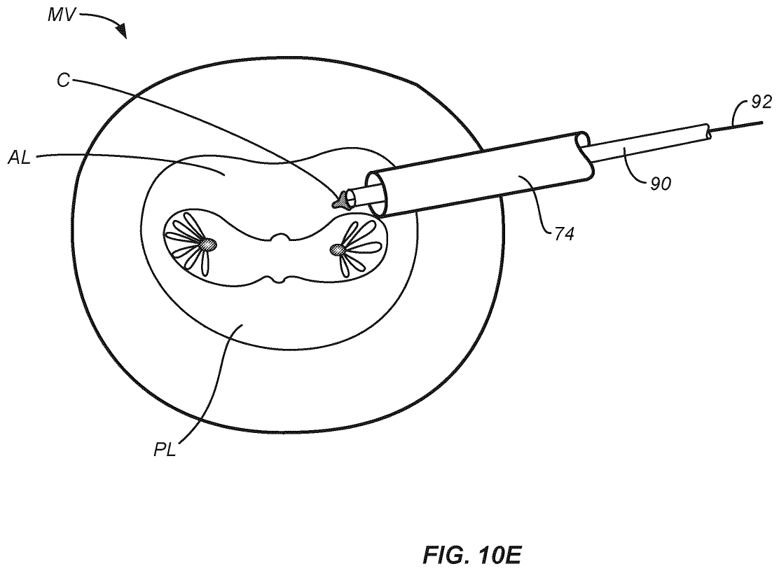

Referring now to FIGS. 10A through 10E, a single valve clip C can be removed from a mitral valve MV by introducing the capture catheter 10 and the transfer catheter 34 into the left atrium using a transseptal sheath 74, as generally described previously. In addition, a clip removal catheter 90 can be introduced through the transseptal sheath 74 simultaneously with the capture catheter 10 and the transfer catheter 34 in order to engage and stabilize the clip C. As shown in FIG. 10A, a clip capture element 94 at the distal end of a manipulation wire 92 can be delivered through a lumen of the clip removal catheter 90 to capture an upper surface of the clip C. The cutting region 54 of the cutting number 50 is then used to excise the tissue in the anterior leaflet AL, while the clip remains held and stabilized by the clip removal catheter 90, thus freeing the clip from the anterior leaflet, as shown in FIG. 10B (where the capture catheter 10 and transfer catheter 34 are removed for ease of illustration). The capture catheter 34 and transfer catheter 10 may then be repositioned, as shown in FIG. 10C, so that the cutting region 54 of the cutting member 50 is positioned against the posterior leaflet PL, and the cutting region can then be manipulated to excise tissue in the posterior leaflet to release the clip, as shown in FIG. 10D. The clip removal catheter 90, which continues to hold the clip C, can be then be used to withdraw the clip C from the mitral valve, as shown in FIG. 10E. The mitral valve MV is then completely free from implanted clips to receive a mitral valve prosthesis or to have another corrective intervention performed.

Although certain embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially.

What is claimed is:

1. A system for excising a clip implanted in valve leaflet tissue, said system comprising:
a transfer catheter having a proximal end and a distal end;
a capture catheter having a distal end, the transfer catheter and the capture catheter both being configured to be introduced into a heart chamber; and
a severing mechanism having a proximal end and a distal end, the severing mechanism configured to be deployed from the transfer catheter toward the capture catheter, the severing mechanism comprising a cutting region comprised a conductive material disposed therein, the conductive region being configured to engage the leaflet tissue to excise the clip.

2. The system as in claim 1, wherein the capture catheter comprises a clip engaging feature carried by the capture catheter, the clip engaging feature being configured to extend distally and radially outward from the capture catheter.

3. The system as in claim 2, wherein the clip engaging feature being configured to engage the clip, the clip engaging featured comprising at least one selected from the group comprising a magnet, a loop, a hook, a coil, a snare, a barb, forceps, jaws, a funnel-shaped receptacle, and a dock.

4. The system of claim 1, further comprising one selected from a group consisting of a magnetic element disposed at the distal end of both the transfer catheter and the capture catheter and a loop on the distal end of the capture catheter configured to capture the distal end of the severing mechanism.

5. The system of claim 1, wherein the severing mechanism comprises one selected from a group consisting of a sharpened member, an abrasive member, ablation member, and an RF electrode member, all being configured to be disposed in the cutting region.

6. The system of claim 1, wherein the severing mechanism is configured to be passed from the transfer catheter to the capture catheter, wherein at least a portion of the cutting region spans the valve leaflet tissue to be excised.

7. The system of claim 1, further comprising a clip removal catheter configured to constrain the implanted clip during the excision.

8. The system of claim 9, wherein the clip removal catheter further being configured to capture and remove the excised clip.

9. The system of claim 1, wherein the cutting region of the severing mechanism is proximate to a middle portion of the severing mechanism.

10. A method for excising a clip implanted in valve leaflet tissue, said method comprising:
introducing a first catheter into a heart chamber adjacent to the valve leaflet tissue on one side of the implanted clip;
introducing a second catheter into the heart chamber adjacent to the valve tissue on another side of the implanted clip;
deploying a severing mechanism from the second r catheter to the first catheter to place at least a portion of the severing mechanism against leaflet tissue, the severing mechanism comprising a cutting region having conductive material disposed therein;
engaging the cutting region with the leaflet tissue; and
excising the clip from the leaflet tissue using the severing mechanism.

11. A method as in claim 17, wherein introducing one of the first catheter or second catheter further comprises steering a distal tip of least one of the capture catheter and the transfer catheter to bring the distal tips of both catheters into proximity with the embedded clip.

12. A method as in claim 17, wherein deploying the severing mechanism further comprises engaging at least a portion of the capture catheter with the distal end of the severing mechanism.

13. A method as claim 13, advancing at least one of the first catheter or second catheter through an opening between the valve leaflet tissue adjacent to the imbedded clip.

14. A method as recited in claim 13, wherein deploying the severing mechanism comprises passing the severing mechanism between the first catheter and the second catheter to span a target region of the valve leaflet to be excised.

15. A method as in claim 13, further comprising extending the severing mechanism past a side of the valve clip so that the severing mechanism is positioned to excise a portion of the valve leaflet tissue on one side of the valve.

16. A method as in claim 13, repositioning the severing mechanism on the other side of the lip so that the opposed leaflet may be excised from the heart.

17. A method as in claim 13, further comprises removing the excised clip from the heart chamber using the capture catheter or a clip removal catheter.

18. A method as in claim 13, further comprising deploying a clip removal catheter into the heart chamber proximate to the clip being excised from the valve leaflet tissue to engage, capture, constrain, or remove the clip being excised from the leaflet tissue.

19. A device for excising a clip implanted in valve leaflet tissue, the device comprising:
an elongate body configured for introduction into a heart chamber, the elongate body having a proximal end and a distal end;
a clip engaging feature carried by the elongate body, the clip engaging feature being configured to extend distally and radially outward from the elongated body, the clip engaging feature configured to engage the clip, the clip engaging featured comprising at least one selected from the group comprising a magnet, a loop, a hook, a coil, a snare, a barb, forceps, jaws, a funnel-shaped receptacle, and a dock; and
a severing mechanism comprising a distal end, the severing mechanism being non-concentrically carried by the elongate body alongside the clip engaging feature, the severing mechanism being configured to extend distally and radially outward from the distal end of the elongate body, the severing mechanism comprising a cutting region comprising a conductive material configured to sever the valve leaflet tissue, the severing mechanism further comprising a radiopaque marker disposed proximal to the exposed cutting region.

20. The device of claim 19, wherein the clip engaging feature comprises a collapsed delivery configuration and an extended deployment configuration.
